(19) United States
(12) Plant Patent Application Publication
(10) Pub. No.: US 2012/0240297 P1
(43) Pub. Date: Sep. 20, 2012

(54) Strawberry plant named 'Ischia'

(50) ;

(75) Inventors: Michelangelo Leis, Ferrara (IT); Alessio Martinelli, Ferrara (IT); Gianfranco Castagnoli, Quingentole (IT); Donata Azzolini, Quingentole (IT); Pietro Castagnoli, Quingentole (IT); Alessandro Castagnoli, Quingentole (IT)

(73) Assignee: C.I.V. - Consorzio Italiano Vivaisti - Società Consertile A R.L.

(21) Appl. No.: 13/385,297

(22) Filed: Feb. 13, 2012

(30) Foreign Application Priority Data
Feb. 14, 2011 (QZ) 2011/0358

(51) Int. Cl. A01H 5/00

(52) U.S. Cl. PLT/208

(57) ABSTRACT

A new and distinct variety of Fragaria L. named ‘ISCHIA’, characterized by the description herein.

### LATIN NAME OF THE GENUS AND SPECIES CLAIMED

Fragaria L.

### VARIETY DENOMINATION

‘ISCHIA’

### PRIORITY CLAIM

This application claims the benefit of European Community Plant Variety Office Plant Breeders' Rights Application No. 2011/0358, filed Feb. 14, 2011.

### BACKGROUND OF THE INVENTION

The present invention relates to a new and distinct strawberry variety named ‘ISCHIA’, which was the result of a controlled cross between the strawberry variety ‘CIVRI30’ (Breeder reference, unpatented) and a not released selection of Consorzio Italiano Vivaisti named O2N2-19.

The progeny was first asexually propagated by stolons in S. Giuseppe di Comacchio Ferrara, Italy, in 2005.

The ‘ISCHIA’ variety was tested over the next several years in different European areas with continental climates. The tests started in 2006 and continued until 2010.

### BRIEF SUMMARY OF THE INVENTION

Plants of the new Strawberry variety ‘ISCHIA’ can be distinguished from plants of the Strawberry variety ‘CIVRI30’ by the following unique combination of characteristics described in Table 1:

TABLE 1
Comparison with Comparison Variety

| | Characteristic in | State of | State of |
|---|---|---|---|
| Denomination of | which the similar | expression of | expression of |
| similar variety | variety is different | similar variety | candidate variety |
| CIVRI30 | Plant Vigor | Weak to Medium | Strong |
| | Leaf Size | Small to Medium | Medium to Large |
| | Fruit Color | Orange Red | Medium Red |

Asexual reproduction of this new variety by stolons shows that the foregoing and all other characteristics and distinctions come true to form and are established and transmitted through succeeding propagations. The new variety reproduces true to type.

### BRIEF DESCRIPTION OF THE PHOTOGRAPHS

The accompanying photographs illustrate the overall appearance of the new Strawberry ‘ISCHIA’ showing the colors as true as is reasonably possible with colored reproductions of this type. Colors in the photographs may differ slightly from the color value cited in the detailed botanical description, which accurately describe the color of ‘ISCHIA’.

FIG. 1 shows the plant of ‘ISCHIA’;

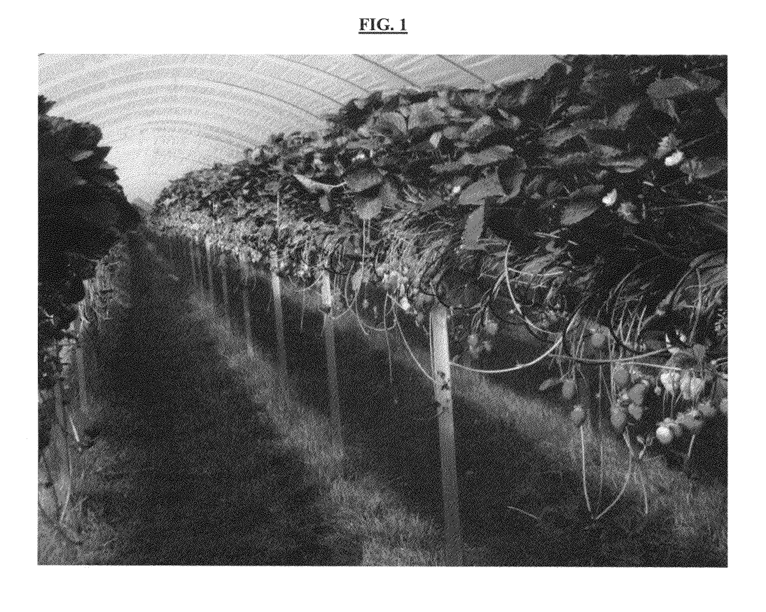

FIG. 2 shows the leaves of ‘ISCHIA’;

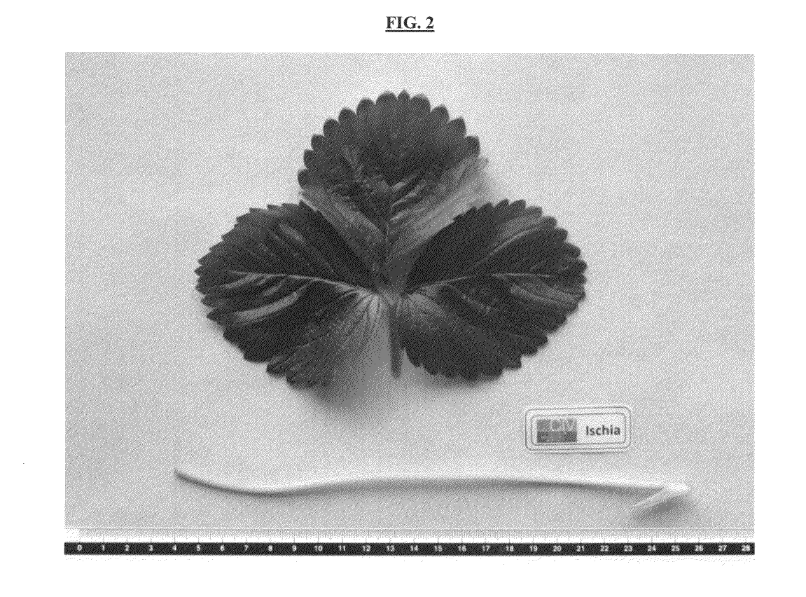

FIG. 3 shows the flowers of ‘ISCHIA’; and

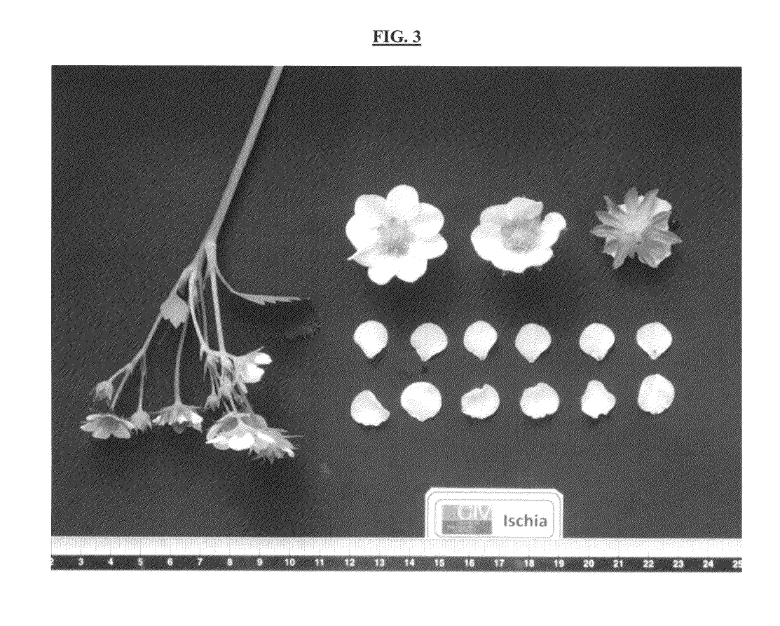

FIG. 4 shows the fruits of ‘ISCHIA’.

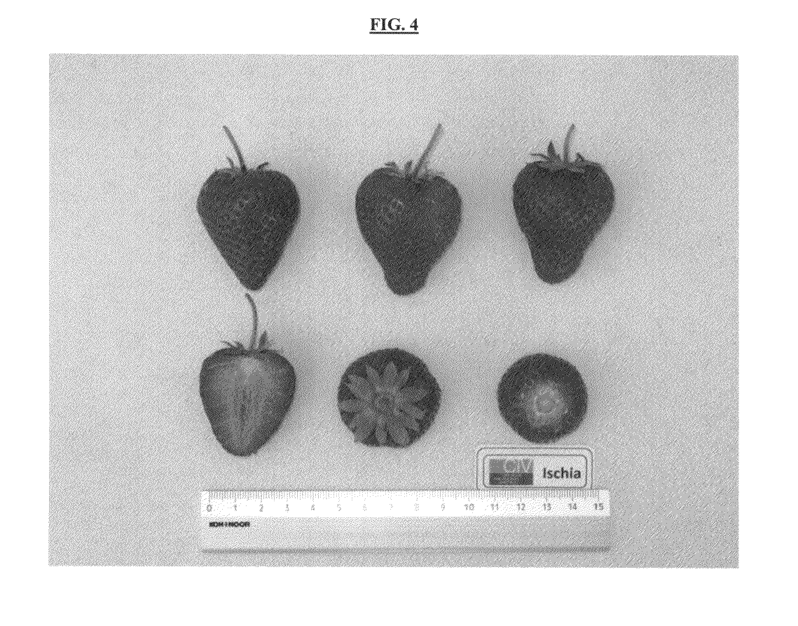

The colors of this illustration may vary with lighting conditions and, therefore, color characteristics of this new variety should be determined with reference to the observations described herein, rather than from these illustrations alone.

### DETAILED BOTANICAL DESCRIPTION

The following detailed description of the ‘ISCHIA’ variety, unless otherwise noted, is based on observations taken of plants and fruits, and asexually reproduced progeny, grown at S. Giuseppe di Comacchio, Ferrara, Italy.

The observed plants were one year old plants. The plants used in the production field are produced in a nursery in S. Giuseppe di Comacchio, dug in December, cold stored, and planted in production fields in the middle of April.

Certain characteristics of this variety may change with changing environmental conditions (e.g., light, temperature, moisture, etc.), nutrient availability, or other factors. Quantified measurements are expressed as an average of measurements taken from a number of individual plants of the new variety. The measurements of any individual plant, or any group of plants, of the new variety may vary from the stated average. Color descriptions and other terminology are used in accordance with their ordinary dictionary descriptions, unless the context clearly indicates otherwise. Color designations are made with reference to The Royal Horticultural Society (R.H.S.) Color Chart.
Plant:
   Habit.—Semi-upright.
   Density.—Dense.
   Vigor.—Medium.
   Overall shape.—Semi-upright.
   Height.—0.27-0.30 meters.
   Width.—0.35-0.37 meters.
Leaf:
   Upperside.—RHS 139A green group; underside: RHS 147B yellow green group.
   Size of leaf.—Medium.
   Cross section.—Concave.
   Leaf surface ondulation or blistering.—Medium.
   Number of leaflets.—Three only.
   Leaf stem characteristics.—Color RHS 145A yellow-green group. Position of hairs: upwards. Density of hairs: Sparse. Length: About 19 cm.
   Terminal leaflet.—Length/width ratio: Moderately Longer. Length: About 7 to 8 cm. Width: About 6 to 7 cm. Shape of base: Obtuse. Shape of teeth: Serrate to Crenate.
   Petiole.—Position of hairs: Upwards. Density of hairs: Sparse. Length: About 19 cm.
   Stipule.—Color RHS 144D yellow-green group; Anthocyanin coloration: absent.
   Stolons.—Number about 5-6. Thickness: Medium, about 2.85 mm. Pubescence: Sparse. Color RHS 145A, yellow-green.
Inflorescence: Position relative to foliage: at same level or slightly below. Length: About 13-15 cm.
   Flower.—Size. Medium. Size of calyx relative to corolla: Same size. Spacing of petals. Overlapping.
   Flower characteristics.—Diameter primary flowers. About 3.2 cm. Diameter secondary flowers. About 2.5 cm. Number of petals: Normally about 5 to 7. Fragrance: No significant fragrance. Time from bloom to mature fruit: About 35 to 38 days.
Reproductive organs:
   Stamens.—Numerous with pollen present, fertile and abundant. Length: approximately 3 mm. Color: near RHS 157A, green-white.
   Anthers.—Generally average in size Length: About 1.2 mm. Width: About 1 mm. Color: RHS 14 B yellow-orange; darkening with advanced maturity.
   Pollen.—Fertile and abundant. Color: RHS 17 A yellow-orange.
   Pistils.—Numerous, generally average in size. Color: RHS 151D yellow-green.
   Petal.—Length/width ratio: Equal.
Fruit:
   Fruit truss.—Attitude: Semi-erect.
   Ratio of length/maximum width.—Moderately longer.
   Color of mature fruit.—RHS 46A to 46B red.
   Color of internal fruit.—RHS 34A orange-red, RHS 33B orange-red, and RHS 155D white.
   Length.—40-50 mm.
   Width.—37-45 mm.
   Calyx diameter.—25-30 mm.
   Average weight.—20-31 grams.
   Achene color.—RHS 152C yellow green.
   Number of achenes per berry.—480-520.
   Achene size.—length about 1.4 mm, width about 1 mm.
   Weight of 1000 achenes.—0.40 grams.
   Peduncle length of inflorescence stem.—Primary fruit about 9-11 cm, secondary fruit about 6-7 cm, color near RHS 139D green.
   Marketable yield (gm/plant).—about 800-1000 grams.
   Fruit size.—large.
   Predominant shape.—Conical.
   Difference in shapes between primary and secondary fruit.—Slight.
   Band without achenes.—absent to very narrow.
   Unevenness of surface.—even or very slightly uneven.
   Evenness of color.—even or very slightly uneven.
   Glossiness.—medium.
   Insertion of achenes.—Level with surface.
   Insertion of calyx.—inserted.
   Attitude of the calyx.—outwards.
   Size of calyx in relation to fruit diameter.—slightly smaller.
   Adherence of calyx.—medium.
   Firmness of flesh.—firm.
   Hollow center expression.—absent or small.
   Flavor.—good.
   Soluble solids (% brix).—8.5-10.
   Time of first flowering.—about 28-32 days after planting using cold stored plants quality A.
   Time of first harvesting.—about 35-38 days after first flowering.
   Harvest period.—June to November.
   Type of bearing.—fully remontant.
Disease resistance: The ‘ISCHIA’ variety has been tested for several years and it has not shown particular problems in cultivation. No specific testing for disease resistances has been carried out.

What is claimed is:

1. A new and distinct variety of Fragaria L. named ‘ISCHIA’, as illustrated and described herein.
